(19) United States
(12) Patent Application Publication
(10) Pub. No.: US 2024/0401139 A1
(43) Pub. Date: Dec. 5, 2024

(54) ASSESSING GRAFT SUITABILITY FOR TRANSPLANTATION

(71) Applicant: Natera, Inc., San Carlos, CA (US)

(72) Inventors: Aoy Tomita MITCHELL, Wauwatosa, WI (US); Michael MITCHELL, Wauwatosa, WI (US)

(73) Assignee: Natera, Inc., San Carlos, CA (US)

(21) Appl. No.: 18/756,518

(22) Filed: Jun. 27, 2024

Related U.S. Application Data
(63) Continuation of application No. 16/954,183, filed on Jun. 15, 2020, now Pat. No. 12,084,720.
(60) Provisional application No. 62/599,011, filed on Dec. 14, 2017.

(51) Int. Cl. C12Q 1/6883

(52) U.S. Cl. CPC C12Q 1/6883; C12Q 2600/118

(57) ABSTRACT

This invention relates to methods and compositions for assessing the suitability of a graft for transplantation or implantation by measuring total and/or specific cell-free nucleic acids (such as cf-DNA) and/or cell lysis. Specifically, the method comprising obtaining an amount of total of DNA and/or graft-specific cfDNA released from a potential graft (e.g., ex vivo), e.g., prior to contacting of the potential graft with blood cells of a potential recipient, and/or subsequent to contacting of the potential graft or cells thereof with blood cells from a potential recipient, assessing the amount(s) to determine the suitability of the potential graft for transplantation or implantation.

### RELATED APPLICATION

This application is a national stage filing under 35 U.S.C. § 371 of International Patent Application No. PCT/US2018/065845, filed Dec. 14, 2018, which claims the benefit under 35 U.S.C. § 119 of the filing date of U.S. Provisional Application No. 62/599,011, filed Dec. 14, 2017, the contents of each of which are incorporated herein by reference in their entirety.

### FIELD OF THE INVENTION

This invention relates to methods and related compositions for assessing the suitability of a graft for transplantation or implantation by measuring total and/or graft-specific cell-free nucleic acids, such as cell-free DNA.

### SUMMARY OF INVENTION

In one aspect, a method of assessing the suitability of a graft is provided.

In one embodiment of any one of the methods provided herein, the method further comprises obtaining the one or more samples.

In one embodiment of any one of the methods provided herein, the value for the amount of total cell-free nucleic acids (such as DNA) and/or value for the amount of specific cell-free nucleic acids (such as DNA) are provided in a report. In one aspect, a report with one or more of the values obtained by any one of the methods provided herein is provided.

In one embodiment, any one of the methods provided can further comprise obtaining a value for the amount of total cell-free nucleic acids (such as DNA) in one or more other samples, and/or obtaining a value for the amount of specific cell-free nucleic acids (such as DNA) in one or more other samples, wherein the one or more other samples are from a subsequent time point or points.

In one embodiment of any one of the methods provided herein, the one or more samples and/or one or more other samples are obtained within minutes, such as no more than 15, 20, 25, 30, 35, 40, 45, 50, or 55 minutes, of obtaining the graft (e.g., storing the graft, perfusing the graft, etc.).

In one embodiment of any one of the methods provided herein, the one or more samples and/or one or more other samples are obtained within hours, such as no more than 1, 2, 3, 4, 5, 6, 7, 8, 9, 10, 12, 18 or more hours, of obtaining the graft (e.g., storing the graft, perfusing the graft, etc.).

In one embodiment of any one of the methods provided herein, an initial sample is obtained within an hour of obtaining the graft and one or more other samples are obtained within 15, 20, 25, 30, 35, 40, 45, 50, or 55 minute intervals or 1, 2, 3, 4, 5, 6, 7, 8, 9, 10, 12, 18 or more hourly intervals, such as until a threshold value or baseline is reached.

In one embodiment of any one of the methods provided herein, the one or more other subsequent time points are at hourly intervals. In one embodiment of any one of the methods provided herein, the one or more other subsequent time points are at daily intervals. In one embodiment of any one of the methods provided herein, the one or more other subsequent time points are at one-week intervals. In one embodiment of any one of the methods provided herein, the one or more other subsequent time points are at two-week intervals. In one embodiment of any one of the methods provided herein, the one or more other subsequent time points are at monthly intervals.

In one embodiment of any one of the methods provided herein, the specific cell-free nucleic acids (such as DNA) are graft-specific cell-free nucleic acids (such as DNA).

In one embodiment of any one of the methods provided herein, the method further comprises obtaining the one or more samples and/or one or more other samples. In one embodiment of any one of the methods provided herein, the method further comprises providing the one or more samples.

In one embodiment of any one of the methods provided herein, the monitoring of the graft comprises any one of the methods provided herein.

In one embodiment of any one of the methods provided herein, the sample comprises media, blood, plasma or serum.

In one aspect, a report comprising any one or more of the values provided herein is provided. In one embodiment of any one of the reports provided, the report comprises a value for the amount of total cell-free nucleic acids (such as DNA) in one or more samples and/or a value for the amount of specific cell-free nucleic acids (such as DNA) in one or more samples.

In one embodiment of any one of the reports provided, the report further comprises a value for the amount of total cell-free nucleic acids (such as DNA) from one or more other samples and/or a value for the amount of specific cell-free nucleic acids (such as DNA) from one or more other samples, wherein the one or more other samples are from a subsequent time point or points. In one embodiment of any one of the reports provided, the subsequent time point is at least one day later. In one embodiment of any one of the reports provided, the subsequent time point is at least one week later. In one embodiment of any one of the reports provided, the subsequent time point is at least two weeks later. In one embodiment of any one of the reports provided, the subsequent time point is at least a month later.

In one embodiment of any one of the methods provided herein, the method for obtaining an amount of total cell-free nucleic acids (such as DNA) comprises amplification, such as with real-time PCR or digital PCR. In one embodiment of any one of such methods comprising amplification, such as with real-time PCR or digital PCR, one or more targets are amplified. In one embodiment of any one of these methods, RNase P is the target or one of the targets for amplification. Any of a number of reference genes can be amplified for the analysis. Other reference genes that can serve as the target for amplification will be known to those of ordinary skill in the art.

In one embodiment of any one of such methods provided herein, the methods for obtaining an amount of specific cell-free nucleic acids (such as DNA) (for example, when a graft is a xenograft) comprises amplification, such as with real-time PCR. In one embodiment of any one of such methods, the method comprises, obtaining a quantification of one or more targets specific to the graft and one or more targets specific to the recipient or potential recipient. In one embodiment of any one of the methods provided herein, the method further comprises obtaining the one or more graft-specific targets and/or the one or more recipient or potential recipient targets. In one embodiment of any one of the methods provided herein, the quantification is obtained for each target relative to a standard, such as an internal standard, that may be spiked into a sample(s).

In one embodiment of any one of such methods provided herein, the methods for obtaining an amount of specific cell-free nucleic acids (such as DNA) can comprise a mismatch PCR amplification method. In one embodiment of any one of the methods provided herein, such a mismatch method comprises, for each of a plurality of single nucleotide variant (SNV) targets, obtaining results from an amplification-based quantification assay, such as a polymerase chain reaction (PCR) quantification assay, on a sample, or portion thereof, with at least one primer pair, wherein the at least one primer pair comprises a forward primer and a reverse primer, wherein the at least one primer pair comprises a primer with a 3′ mismatch (e.g., penultimate mismatch) relative to one sequence (e.g., allele) of the SNV target but a 3′ double mismatch relative to another sequence (e.g., allele) of the SNV target and specifically amplifies the one sequence (e.g., allele) of the SNV target.

In one embodiment of any one of the methods provided herein, such a mismatch method further comprises, for each SNV target, obtaining results from a quantification assay with at least one another primer pair, wherein the at least one another primer pair comprises a forward primer and a reverse primer, wherein the at least one another primer pair specifically amplifies another sequence (e.g., allele) of the SNV target.

In one embodiment of any one of the methods provided herein, such a mismatch method comprises, for each of a plurality of single nucleotide variant (SNV) targets, performing an amplification-based quantification assay, such as a PCR quantification assay, on a sample, or portion thereof, with at least two primer pairs, wherein each primer pair comprises a forward primer and a reverse primer, wherein one of the at least two primer pairs comprises a 3′ mismatch (e.g., penultimate) relative to one sequence (e.g., allele) of the SNV target but a 3′ double mismatch relative to another sequence (e.g., allele) of the SNV target and specifically amplifies the one sequence (e.g., allele) of the SNV target, and another of the at least two primer pairs specifically amplifies the another sequence (e.g., allele) of the SNV target.

In one embodiment of any one of the methods provided herein, such a mismatch method comprises obtaining results from an amplification-based amplification assay, such as a polymerase chain reaction (PCR) quantification assay, for each of a plurality of single nucleotide variant (SNV) targets, performed on a sample, or portion thereof, with at least two primer pairs, wherein each primer pair comprises a forward primer and a reverse primer, wherein one of the at least two primer pairs comprises a 3′ mismatch (e.g., penultimate) relative to one sequence (e.g., allele) of the SNV target but a 3′ double mismatch relative to another sequence (e.g., allele) of the SNV target and specifically amplifies the one sequence (e.g., allele) of the SNV target, and another of the at least two primer pairs specifically amplifies the another sequence (e.g., allele) of the SNV target.

In one embodiment of any one of the methods provided herein, such a mismatch method comprises obtaining results from an amplification-based quantification assay, such as a polymerase chain reaction (PCR) assay on a sample with at least one primer pair as provided herein, such as at least two primer pairs, wherein each primer pair comprises a forward primer and a reverse primer, selecting informative results based on the genotype of the specific nucleic acids and/or non-specific nucleic acids, and determining the amount of the non-specific nucleic acids in the sample based on the informative results. In one embodiment of any one of the methods provided herein, such a mismatch method further comprises identifying the plurality of SNV targets. In one embodiment of any one of the methods provided herein, such a mismatch method further comprises inferring the genotype of the non-specific nucleic acids.

In one embodiment of any one of the methods provided herein, such a mismatch method comprises obtaining results from 1) an amplification-based quantification assay, such as a PCR quantification assay, for each of a plurality of SNV targets, performed on a sample, or portion thereof, with at least one primer pair, such as at least two primer pairs, wherein each primer pair comprises a forward primer and a reverse primer, wherein one of the at least one, such as at least two, primer pair, comprises a 3′ mismatch (e.g., penultimate) relative to one sequence (e.g., allele) of the SNV target but a 3′ double mismatch relative to another sequence (e.g., allele) of the SNV target and specifically amplifies the one sequence (e.g., allele) of the SNV target and 2) a determination of informative results based on the specific genotype and/or a prediction of the likely non-specific genotype. In one embodiment of any one of such mismatch methods, when there are at least two primer pairs, the another primer pair specifically amplifies the another sequence (e.g., allele) of each SNV target and quantification results are obtained with the another primer pair for each of the SNV targets.

In one embodiment of any one of the methods provided herein, such a mismatch method comprises obtaining results from 1) an amplification-based quantification assay, such as a PCR quantification assay, for each of a plurality of SNV targets, performed on a sample, or portion thereof, with at least two primer pairs, wherein each primer pair comprises a forward primer and a reverse primer, wherein one of the at least two primer pairs comprises a 3′ mismatch (e.g., penultimate) relative to one sequence (e.g., allele) of the SNV target but a 3′ double mismatch relative to another sequence (e.g., allele) of the SNV target and specifically amplifies the one sequence (e.g., allele) of the SNV target, and another of the at least two primer pairs specifically amplifies the another sequence (e.g., allele) of the SNV target, and 2) a determination of informative results based on the specific genotype and/or a prediction of the likely non-specific genotype.

In one embodiment of any one of the methods provided herein, such a mismatch method further comprises at least one another primer pair for each SNV target and/or obtaining results with an amplification-based quantification assay, such as a PCR quantification assay therewith. In one embodiment of any one of such mismatch methods, the at least one another primer pair comprises a 3′ mismatch (e.g., penultimate) relative to another sequence (e.g., allele) of the SNV target but a 3′ double mismatch relative to the one sequence (e.g., allele) of the SNV target and specifically amplifies the another sequence (e.g., allele) of the SNV target.

In one embodiment of any one of the methods provided herein, such a mismatch method further comprises assessing the amount of specific nucleic acids based on the results.

In one embodiment of any one of such mismatch methods, the results are informative results.

In one embodiment of any one of such mismatch methods, the method further comprises selecting informative results of the amplification-based quantification assays, such as PCR quantification assays. In one embodiment of any one of such mismatch methods, the selected informative results are averaged, such as a median average. In one embodiment of any one of such mismatch methods, the results can be further analyzed with Robust Statistics. In one embodiment of any one of such mismatch methods, the results can be further analyzed with a Standard Deviation, such as a Robust Standard Deviation, and/or Coefficient of Variation, such as a Robust Coefficient of Variation, or % Coefficient of Variation, such as a % Robust Coefficient of Variation.

In one embodiment of any one of such mismatch methods, the informative results of the amplification-based quantification assays, such as PCR quantification assays are selected based on the genotype of the non-specific nucleic acids and/or specific nucleic acids.

In one embodiment of any one of such mismatch methods, the method further comprises obtaining the genotype of the non-specific nucleic acids and/or specific nucleic acids.

In one embodiment of any one of such mismatch methods, there is at least one primer pair, at least two primer pairs, at least three primer pairs, at least four primer pairs or more per SNV target. In one embodiment of any one of such mismatch methods, the plurality of SNV targets is at least 45, 48, 50, 55, 60, 65, 70, 75, 80, 85 or 90 or more. In one embodiment of any one of such mismatch methods, the plurality of SNV targets is at least 90, 95 or more targets. In one embodiment of any one of such mismatch methods, the plurality of SNV targets is less than 90, 95 or more targets. In one embodiment of any one of such mismatch methods, the plurality of SNV targets is less than 105 or 100 targets.

In one embodiment of any one of such mismatch methods, the mismatched primer(s) is/are the forward primer(s). In one embodiment of any one of such mismatch methods, the reverse primers for the primer pairs for each SNV target is the same.

In one embodiment of any one of the methods provided herein, the amount of the specific cell-free nucleic acids (such as DNA) is the ratio or percentage of specific nucleic acids to total or non-specific nucleic acids.

In one embodiment of any one of the methods provided herein, the method further comprises extracting nucleic acids from the sample.

In one embodiment of any one of the methods provided herein, the method further comprises a pre-amplification step. In one embodiment of any one of the methods provided herein, the pre-amplification is performed prior to the quantification assay(s).

In one embodiment, any one of the embodiments for the methods provided herein can be an embodiment for any one of the reports provided. In one embodiment, any one of the embodiments for the reports provided herein can be an embodiment for any one of the methods provided herein.

### BRIEF DESCRIPTION OF FIGURES

The accompanying figures are not intended to be drawn to scale. The figures are illustrative only and are not required for enablement of the disclosure.

FIG. 1 provides an exemplary, non-limiting diagram of MOMA primers. In a polymerase chain reaction (PCR) assay, extension of the sequence containing SNV A is expected to occur, resulting in the detection of SNV A, which may be subsequently quantified. Extension of the SNV B, however, is not expected to occur due to the double mismatch.

FIG. 2 shows exemplary outline for determining cell-free nucleic acids (such as DNA).

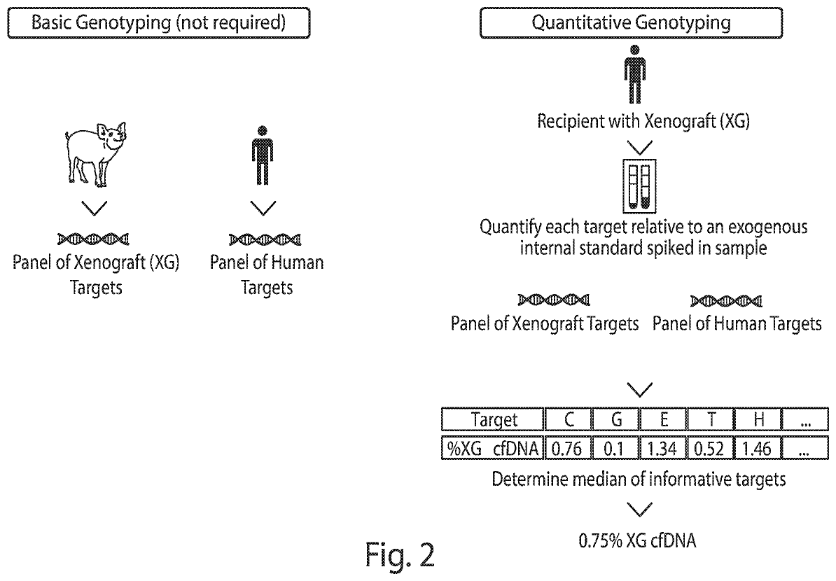

FIG. 3 is a workflow schematic depicting one embodiment of a sample processing scheme.

FIG. 4 shows the proportion of long fragment DNA in the eluates (reported as the mean of duplicate determinations) in the control (normal plasma specimen) and tubes 2-5.

FIG. 5 is a representative electropherogram image of a fragment distribution in Tubes 2-5. The peak height indicates the fragment levels. The fragment length (BP) is indicated on the trace. FU=fluorescence units.

FIG. 6 shows the STEEN/plasma supernatant after 1400×g spin, showing essential absence of leukocytes, similar to that seen in plasma collections in the absence of STEEN (magnification 400×) (left panel). For comparison, the resuspended 200 μL 1400×g pellet (magnification 400×) showing effective removal of any residual leukocytes in the post-PPT STEEN/plasma supernatant by the subsequent 1400×g spin step, congruent with results seen using plasma without STEEN (right panel).

FIG. 7 is a graph showing the percent recovery of different concentrations of gDNA and heparin added to STEEN™ solution.

### DETAILED DESCRIPTION OF THE INVENTION

Total cell free DNA of a graft ex vivo in a perfusion container generally will represent lysis and/or apoptosis of cells from the graft and any cells from blood from the donor. Without being bound by theory, it is thought that as a graft starts to deteriorate, apoptosis of cells increase, and total cell free DNA levels will also increase. A suitable graft for transplant generally has low or steady-state levels of total cell free DNA.

Aspects of the disclosure relate to methods for assessing the suitability of a graft. Methods provided herein or otherwise known in the art can be used multiple times to obtain total and/or specific cell-free nucleic acid (such as DNA) values over time. Also included are reports that can include one or more of these values. Such reports can provide valuable information to a clinician. In some embodiments, the clinician can then assess the condition (or suitability of a graft) and/or make treatment decisions accordingly for a subject.

As used herein, “graft” refers to a biological material comprising cells or tissue, such as at least a portion of an organ, that may be transplanted or implanted in or into a subject. In some embodiments, the graft is explanted material comprising cells or tissue, such as at least a portion of an organ that is being maintained outside the body (ex vivo), such as to preserve or rehabilitate, the graft. Any one of the methods provided herein can be used to evaluate its suitability for future engraftment. In one embodiment of any one of the methods provided herein, the material are EVLP lungs, such as after removal from a subject and before engraftment into another subject.

In some embodiments of any one of the methods provided herein, the graft is a whole organ or more than one organ. Examples of organs that can be transplanted or implanted include, but are not limited to, the heart, kidney(s), kidney, liver, lung(s), pancreas, intestine, etc. An example of more than one organ includes a combination of a heart and lung. In other embodiments of any one of the methods provided herein, the graft is less than a whole organ and is at most a portion thereof, such as a valve. Grafts may be of the same species or may be of a different species.

Accordingly, in some embodiments of any one of the methods provided herein, the graft is from a different species (or is a xenograft), such as from a pig or cow when the recipient is other than a pig or cow, respectively, such as a human. Any one of the types of grafts provided herein may be a xenograft. In some embodiments of any one of the methods provided herein, the graft is a pig or cow valve. In other embodiments of any one of the methods provided herein, the graft is from the same species. In other embodiments of any one of the methods provided herein the graft is decellularized graft, such as a decellularized xenograft. In some embodiments of any one of the methods provided herein the graft is an autograft. Any one of the methods or compositions provided herein may be used for assessing any one of the grafts described herein.

As used herein, the sample can be a biological sample. Examples of such biological samples include whole blood, plasma, serum, etc.

In one embodiment of any one of the methods provided herein, the sample may be of or comprise media in which the graft is placed or with which it has contact. In one embodiment of any one of such samples, the media can comprise blood or a blood substitute, preservation solution, or any other solution in which a graft can be placed or with which it has contact, such as in in vitro contexts. In one embodiment of any one of such samples the graft, such as an organ or organs can be contained in a perfusion system.

In one embodiment of any one of the methods provided herein, the graft (e.g., cells, tissue, organ) is maintained in graft storage media. Graft storage media, such as organ preservation solutions, are well known in the art. Graft storage media can be intracellular (e.g., perfused) or extracellular, and may depend on the graft to be preserved. Approaches to preserving most grafts include simple static cold storage (SCS) and dynamic preservation. Examples of dynamic preservation include hypothermic machine perfusion (HMP), normothermic machine perfusion, and oxygen persufflation. Typically, in combination with hypothermia, graft storage media can prevent clotting in harvests with blood present, reduce stress and deterioration associated with ex vivo handling, and decrease the risk of microbial growth. Therefore, in some embodiments, the graft storage media can comprise osmotic active agents, electrolytes, hydrogen ion buffers, colloid(s), metabolic inhibitors, metabolites, and antioxidants. Examples of osmotic active agents, which may prevent cell swelling, include lactobionate, raffinose, citrate, and gluconate. Electrolytes, which can exert an osmotic effect, include sodium, potassium, calcium, and magnesium ions. Examples of hydrogen ion buffers include phosphate, histidine, and N-(2-hydroxyethyl)-piperazine-N′-2-ethanesulfonic acid (HEPES) buffer. Examples of colloids, which may be used during the initial vascular flush out and perfusion, include albumin and HES. Examples of metabolic inhibitors, which may suppress degradation of cell constituents, include allopurinol, antiproteases, and chlorpromazine. Examples of metabolites, which can help restore metabolism during the reperfusion phase, include adenosine, glutathione, and phosphate. Examples of antioxidants, which can inhibit oxygen free-radical injury, include steroids, vitamin E, deferoxamine, and tryptophan.

Graft storage media are commercially available, and examples include BELZER UW® cold storage solution (VIASPAN™ or the University of Wisconsin (UW) solution), CELSIOR®, CUSTODIOL®, and IGL-1®.

In some aspects, the methods include steps for determining a value for the amount of total cell-free nucleic acids (such as DNA) and/or a value for the amount of specific cell-free nucleic acids (such as DNA).

As used herein, a “value” is any indicator that conveys information about an “amount”. The indicator can be an absolute or relative value for the amount. As used herein, “amount” refers to the quantity of nucleic acids (such as DNA). Further, the value can be the amount, frequency, ratio, percentage, etc.

In some instances the values can be compared to a “threshold value”. As used herein, a “threshold value” refers to any predetermined level or range of levels that is indicative of a state, the presence or absence of a condition or the presence or absence of a risk. The threshold value can take a variety of forms. It can be single cut-off value, such as a median or mean. It can be established based upon comparative groups, such as where the risk in one defined group is double the risk in another defined group. As another example, a threshold value is a baseline value, such as without the presence of a state, condition or risk or after a course of treatment or other remedial action. Such a baseline can be indicative of a normal or other state not correlated with the risk or condition or state that is being tested for.

As used herein, “specific cell-free nucleic acids” refers to a subset of cell-free nucleic acids (such as DNA) that is within total cell-free nucleic acids (such as DNA). In some embodiments, the specific cell-free nucleic acids (such as DNA) are cell-free nucleic acids (such as DNA) that are graft-specific (GS). GS cf-DNA refers to DNA that presumably is shed from the graft or cells thereof, the sequence of which matches (in whole or in part) the genotype of the subject from which the graft is obtained. As used herein, GS cf-DNA may refer to certain sequence(s) in the GS cf-DNA population, where the sequence is distinguishable from the recipient or potential recipient cf-DNA (e.g., having a different sequence at a particular nucleotide location(s)), or it may refer to the entire GS cf-DNA population).

The values for the amount(s) of nucleic acids (such as DNA) can be “obtained” by any one of the methods provided herein, and any obtaining step(s) can include any one of the methods incorporated herein by reference or otherwise provided herein. “Obtaining” as used herein refers to any method by which the respective information or materials can be acquired. Thus, the respective information can be acquired by experimental methods. Respective materials can be created, designed, etc. with various experimental or laboratory methods, in some embodiments. The respective information or materials can also be acquired by being given or provided with the information, such as in a report, or materials. Materials may be given or provided through commercial means (i.e. by purchasing), in some embodiments.

As provided herein, the suitability can be determined using one or more values for the amount of total cell-free nucleic acids (such as DNA) and/or one or more values for the amount of specific cell-free nucleic acids (such as DNA).

Ideally, most of the cell free DNA to be analyzed will come from the organ, and the blood will have washed away. However, intact leukocytes from the donor can still be present in the organ. Also, lysis of cells can lower the quality of the perfusate for total cell free DNA analysis. Thus, in some embodiments of any one of the methods provided herein, contaminating intact cells are removed from samples, such as perfusate samples, by one or more (e.g., one, two or three or more) centrifugation steps. In one embodiment of any one of the methods provided herein, a baseline can be established for meaningful cfDNA analysis after effective washout of contaminating leukocytes.

The suitability can also be determined using one or more values for the amount of total cell-free nucleic acids (such as DNA) and/or one or more values from fragment analysis.

Fragment analysis can be performed by assessing short and/or long nucleic acid fragments. As used herein, a “long fragment” refers to a fragment that is greater than 170 bps (e.g., between 171 and 300 bps in length), while a “short fragment” is a fragment that is less than or equal to 170 bps (e.g., between 75 and 170 bps in length). Such methods generally are performed with primers targeting a long fragment and/or a short fragment.

The fragment can be an Alu fragment. An Alu element is a short stretch of DNA originally characterized by the action of the Arthrobacter luteus (Alu) restriction endonuclease. Alu repeats are the most abundant sequences in the human genome, with a copy number of about 1.4 million per genome. Alu sequences are short interspersed nucleotide elements (SINEs), typically 300 nucleotides, which account for more than 10% of the genome. Provided herein are methods that in one embodiment can include measuring the potential contaminating contribution of cell lysis of a cf-DNA sample by analyzing long Alu fragments and/or short Alu fragments.

In some embodiments of any one of the methods provided, the method further includes assessing the suitability (e.g., health, state, or condition) of a graft for transplantation or implantation based on the value(s). In some embodiments, any one of the methods provided herein can comprise correlating an increase in one or more values (e.g., for an amount of total and/or specific cell-free nucleic acids (such as DNA)) with unsuitability or declining suitability or a decrease in one or more values (e.g., for an amount of total and/or specific cell-free nucleic acids (such as DNA)) with suitability or increasing suitability. In some embodiments of any one of the methods provided herein, correlating comprises comparing a level (e.g., concentration, ratio or percentage) to a threshold value or value from another point in time to determine suitability, or increasing or decreasing suitability. Thus, changes in the levels can be monitored over time. Any one of the methods provided herein can include one or more steps of comparing the values for an amount of nucleic acids (such as DNA) to a threshold value or a value from a different point in time to assess the suitability of the graph.

In one embodiment of any one of the methods provided herein, the method may further includes an additional test(s) for assessing. The additional test(s) may be any one of the methods provided herein or methods known in the art.

It has been found that particularly useful to a clinician is a report that contains the value(s) provided herein. In some embodiments of any one of the reports provided, the reports also include one or more threshold values. In one aspect, therefore such reports are provided. Reports may be in oral, written (or hard copy) or electronic form, such as in a form that can be visualized or displayed. In some embodiments, the “raw” results for each assay as provided herein are provided in a report, and from this report, further steps can be taken to analyze the amount(s) nucleic acids (such as DNA). In other embodiments, the report provides multiple values for the amounts of nucleic acids (such as DNA). From the amounts, in some embodiments, a clinician may assess the suitability of a graft for transplantation or implantation or the need to monitor the graft over time or treatment or some other remedial action.

In some embodiments, the amounts are in or entered into a database. In one aspect, a database with such values is provided. From the amount(s), a clinician may assess the need for a treatment or monitoring. Accordingly, in any one of the methods provided herein, the method can include assessing the amount(s) at more than one point in time. Such assessing can be performed with any one of the methods or compositions provided herein.

In any one of the methods provided herein, the method can include assessing the amount of nucleic acids (such as DNA) at another point in time or times. Such assessing can be performed with any one of the methods provided herein.

Methods for determining total cell-free nucleic acids (such as DNA) as well as specific cell-free nucleic acids (such as DNA) are provided herein or are otherwise known in the art. For example, the methods of PCT Application No. PCT/US2016/030313 may be used for determining a value for the amount of specific cell-free nucleic acids (such as DNA) in a sample as provided herein. Thus, any one of the methods provided herein may include the steps of any one of the methods described in PCT Application No. PCT/US2016/030313, and such methods and steps are incorporated herein by reference. Likewise, the methods of measuring cell-free DNA of U.S. Publication No. US-2015-0086477-A1 are also incorporated herein by reference and such methods can be included as part of any one of the methods provided herein for determining a value for the amount of cell-free nucleic acids (such as DNA).

As a further example, amplification with PCR, such as real-time PCR or digital PCR, may be used to determine a value for the amount of total cell-free nucleic acids (such as DNA) and/or specific cell-free nucleic acids (such as DNA). For example, in some embodiments of any one of the methods provided herein, the total cell-free nucleic acids (such as DNA) is determined with Taqman Real-time PCR using RNase P as a target. Other methods are provided elsewhere herein or would be apparent to those of ordinary skill in the art. Any one of the methods provided herein, can include any one of the methods of determining a value provided herein.

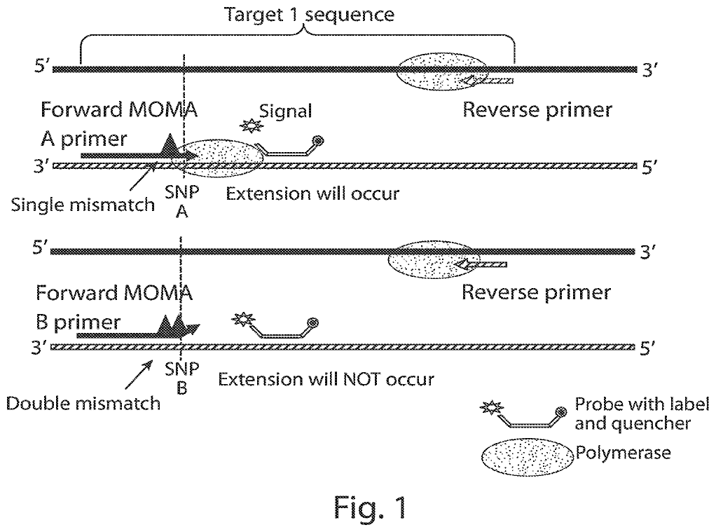

As mentioned above, in some embodiments, any one of the methods provided herein may include steps of a quantitative assay that makes use of mismatch amplification (e.g., MOMA) in order to determine a value for an amount of specific cell-free nucleic acids (such as DNA). Primers for use in such assays may be obtained, and any one of the methods provided herein can include a step of obtaining one or more primer pairs for performing the quantitative assays. Generally, the primers possess unique properties that facilitate their use in quantifying amounts of nucleic acids. For example, a forward primer of a primer pair can be mismatched at a 3′ nucleotide (e.g., penultimate 3′ nucleotide). In some embodiments of any one of the methods provided, this mismatch is at a 3′ nucleotide but adjacent to the SNV position. In some embodiments of any one of the methods provided, the mismatch positioning of the primer relative to a SNV position is as shown in FIG. 1. Generally, such a forward primer even with the 3′ mismatch to produce an amplification product (in conjunction with a suitable reverse primer) in an amplification reaction, thus allowing for the amplification and resulting detection of a nucleic acid with the respective SNV. If the particular SNV is not present, and there is a double mismatch with respect to the other allele of the SNV target, an amplification product will generally not be produced. Preferably, in some embodiments of any one of the methods provided herein, for each SNV target a primer pair is obtained whereby specific amplification of each allele can occur without amplification of the other allele(s).

“Specific amplification” refers to the amplification of a specific target without substantial amplification of another nucleic acid or without amplification of another nucleic acid sequence above background or noise. In some embodiments, specific amplification results only in the amplification of the specific allele.

As used herein, “single nucleotide variant” refers to a nucleic acid sequence within which there is sequence variability at a single nucleotide. In some embodiments, the SNV is a biallelic SNV, meaning that there is one major allele and one minor allele for the SNV. In some embodiments, the SNV may have more than two alleles, such as within a population. Generally, a “minor allele” refers to an allele that is less frequent in a set of nucleic acids, for a locus, while a “major allele” refers to the more frequent allele in a set of nucleic acids. The methods provided herein can quantify nucleic acids of major and minor alleles within a mixture of nucleic acids even when present at low levels, in some embodiments.

The nucleic acid sequence within which there is sequence identity variability, such as a SNV, is generally referred to as a “target”. As used herein, a “SNV target” refers to a nucleic acid sequence within which there is sequence variability at a single nucleotide. The SNV target has more than one allele, and in preferred embodiments, the SNV target is biallelic. In some embodiments of any one of the methods provided herein, the SNV target is a SNP target. In some of these embodiments, the SNP target is biallelic. In some embodiments of any one of the methods provided, the amount of nucleic acids is determined by attempting amplification-based quantitative assays, such as quantitative PCR assays, with primers for a plurality of SNV targets. A “plurality of SNV targets” refers to more than one SNV target where for each target there are at least two alleles. Preferably, in some embodiments, each SNV target is expected to be biallelic and a primer pair specific to each allele of the SNV target is used to specifically amplify nucleic acids of each allele, where amplification occurs if the nucleic acid of the specific allele is present in the sample.

In some embodiments of any one of the methods provided herein, for each SNV target that is biallelic, there are two primer pairs, each specific to one of the two alleles and thus have a single mismatch with respect to the allele it is to amplify and a double mismatch with respect to the allele it is not to amplify (again if nucleic acids of these alleles are present). In some embodiments of any one of the methods provided herein, the mismatch primer is the forward primer. In some embodiments of any one of the methods provided herein, the reverse primer of the two primer pairs for each SNV target is the same.

These concepts can be used in the design of primer pairs for any one of the methods provided herein. It should be appreciated that the forward and reverse primers are designed to bind opposite strands (e.g., a sense strand and an antisense strand) in order to amplify a fragment of a specific locus of the template. The forward and reverse primers of a primer pair may be designed to amplify a nucleic acid fragment of any suitable size to detect the presence of, for example, an allele of a SNV target according to the disclosure. Any one of the methods provided herein can include one or more steps for obtaining one or more primer pairs as described herein.

Generally, “informative results” as provided herein are the results that can be used to quantify the level of nucleic acids in a sample. In some embodiments of any one of the methods provided, the amount of specific- and/or non-specific nucleic acids represents an average across informative results for the nucleic acids, respectively. In some embodiments of any one of the methods provided herein, this average is given as an absolute amount or as a percentage. Preferably, in some embodiments of any one of the methods provided herein, this average is the median.

The amount, such as ratio or percentage, of specific nucleic acids may be determined with the quantities of the major and minor alleles as well as genotype, as needed. In some embodiments of any one of the methods provided herein, the alleles can be determined based on prior genotyping (e.g., of the recipient or potential recipient and/or the subject from which a graft is obtained, respectively). Methods for genotyping are well known in the art. Such methods include sequencing, such as next generation, hybridization, microarray, other separation technologies or PCR assays. Any one of the methods provided herein can include steps of obtaining such genotypes.

It should be appreciated that the primer pairs described herein may be used in a multiplex assays, such as multiplex PCR assays. Accordingly, in some embodiments, the primer pairs are designed to be compatible with other primer pairs in a PCR reaction. For example, the primer pairs may be designed to be compatible with at least 2, at least 5, at least 10, at least 20, at least 30, at least 40, etc. other primer pairs in a PCR reaction. As used herein, primer pairs in a PCR reaction are “compatible” if they are capable of amplifying their target in the same PCR reaction. In some embodiments, primer pairs are compatible if the primer pairs are inhibited from amplifying their target nucleic acid (such as DNA) by no more than 1%, no more than 2%, no more than 5%, no more than 10%, no more than 15%, no more than 20%, no more than 25%, no more than 30%, no more than 35%, no more than 40%, no more than 45%, no more than 50%, or no more than 60% when multiplexed in the same PCR reaction. Primer pairs may not be compatible for a number of reasons including, but not limited to, the formation of primer dimers and binding to off-target sites on a template that may interfere with another primer pair. Accordingly, the primer pairs of the disclosure may be designed to prevent the formation of dimers with other primer pairs or limit the number of off-target binding sites. Exemplary methods for designing primers for use in a multiplex assays are known in the art and are otherwise described herein.

In some embodiments of any one of the methods provided herein, the mismatch amplification-based quantitative assay is any quantitative assay whereby nucleic acids are amplified and the amounts of the nucleic acids can be determined. Such assays include those whereby nucleic acids are amplified with the MOMA primers as described herein and quantified. Such assays include simple amplification and detection, hybridization techniques, separation technologies, such as electrophoresis, next generation sequencing and the like.

In some embodiments of any one of the methods provided herein, the quantitative assays include quantitative PCR assays. Quantitative PCR include real-time PCR, digital PCR, Taqman, etc. In some embodiments of any one of the methods provided herein the PCR is “Real-time PCR”. Such PCR refers to a PCR reaction where the reaction kinetics can be monitored in the liquid phase while the amplification process is still proceeding. In contrast to conventional PCR, real-time PCR offers the ability to simultaneously detect or quantify in an amplification reaction in real time. Based on the increase of the fluorescence intensity from a specific dye, the concentration of the target can be determined even before the amplification reaches its plateau.

In any one of the methods provided herein the PCR may be digital PCR. Digital PCR involves partitioning of diluted amplification products into a plurality of discrete test sites such that most of the discrete test sites comprise either zero or one amplification product. The amplification products are then analyzed to provide a representation of the frequency of the selected genomic regions of interest in a sample. Analysis of one amplification product per discrete test site results in a binary “yes-or-no” result for each discrete test site, allowing the selected genomic regions of interest to be quantified and the relative frequency of the selected genomic regions of interest in relation to one another be determined. In certain aspects, in addition to or as an alternative, multiple analyses may be performed using amplification products corresponding to genomic regions from predetermined regions. Results from the analysis of two or more predetermined regions can be used to quantify and determine the relative frequency of the number of amplification products. Using two or more predetermined regions to determine the frequency in a sample reduces a possibility of bias through, e.g., variations in amplification efficiency, which may not be readily apparent through a single detection assay. Methods for quantifying DNA using digital PCR are known in the art and have been previously described, for example in U.S. Patent Publication number US20140242582.

Any one of the methods provided herein can comprise extracting nucleic acids, such as cell-free DNA. Such extraction can be done using any method known in the art or as otherwise provided herein (see, e.g., Current Protocols in Molecular Biology, latest edition, or the QIAamp circulating nucleic acid kit or other appropriate commercially available kits). An exemplary method for isolating cell-free DNA from blood is described. Blood containing an anti-coagulant such as EDTA or DTA is collected. The plasma, which contains cf-DNA, is separated from cells present in the blood (e.g., by centrifugation or filtering). An optional secondary separation may be performed to remove any remaining cells from the plasma (e.g., a second centrifugation or filtering step). The cf-DNA can then be extracted using any method known in the art, e.g., using a commercial kit such as those produced by Qiagen. Other exemplary methods for extracting cf-DNA are also known in the art (see, e.g., Cell-Free Plasma DNA as a Predictor of Outcome in Severe Sepsis and Septic Shock. Clin. Chem. 2008. v. 54, p. 1000-1007; Prediction of MYCN Amplification in Neuroblastoma Using Serum DNA and Real-Time Quantitative Polymerase Chain Reaction. JCO 2005, v. 23, p.5205-5210; Circulating Nucleic Acids in Blood of Healthy Male and Female Donors. Clin. Chem. 2005, v. 51, p. 1317-1319; Use of Magnetic Beads for Plasma Cell-free DNA Extraction: Toward Automation of Plasma DNA Analysis for Molecular Diagnostics. Clin. Chem. 2003, v. 49, p. 1953-1955; Chiu RWK, Poon LLM, Lau TK, Leung TN. Wong EMC, Lo YMD. Effects of blood-processing protocols on fetal and total DNA quantification in maternal plasma. Clin Chem 2001; 47:1607-1613; and Swinkels et al. Effects of Blood-Processing Protocols on Cell-free DNA Quantification in Plasma. Clinical Chemistry, 2003, vol. 49, no. 3, 525-526).

In some embodiments of any one of the methods provided herein, a pre-amplification step is performed. An exemplary method of such a pre-amplification is as follows, and such a method can be included in any one of the methods provided herein. Approximately 15 ng of cell-free plasma DNA is amplified in a PCR using Q5 DNA polymerase with approximately 13 targets where pooled primers were at 4 uM total. Samples undergo approximately 25 cycles. Reactions are in 25 ul total. After amplification, samples can be cleaned up using several approaches including AMPURE bead cleanup, bead purification, or simply ExoSAP-IT™, or Zymo.

Various aspects of the present invention may be used alone, in combination, or in a variety of arrangements not specifically discussed in the embodiments described in the foregoing and are therefore not limited in their application to the details and arrangement of components set forth in the foregoing description or illustrated in the drawings. For example, aspects described in one embodiment may be combined in any manner with aspects described in other embodiments.

Also, embodiments of the invention may be implemented as one or more methods, of which an example has been provided. The acts performed as part of the method(s) may be ordered in any suitable way. Accordingly, embodiments may be constructed in which acts are performed in an order different from illustrated, which may include performing some acts simultaneously, even though shown as sequential acts in illustrative embodiments.

Use of ordinal terms such as “first,” “second,” “third,” etc., in the claims to modify a claim element does not by itself connote any priority, precedence, or order of one claim element over another or the temporal order in which acts of a method are performed. Such terms are used merely as labels to distinguish one claim element having a certain name from another element having a same name (but for use of the ordinal term).

The phraseology and terminology used herein is for the purpose of description and should not be regarded as limiting. The use of “including,” “comprising,” “having,” “containing”, “involving”, and variations thereof, is meant to encompass the items listed thereafter and additional items.

Having described several embodiments of the invention in detail, various modifications and improvements will readily occur to those skilled in the art. Such modifications and improvements are intended to be within the spirit and scope of the invention. Accordingly, the foregoing description is by way of example only, and is not intended as limiting. The following description provides examples of the methods provided herein.

### EXAMPLES

### Example 1—Evaluation of Gel-Separator Plasma Preparation Tubes (PPT)

Eight mL of neat STEEN™ was added to a 8.5 mL PPT and spun for 10 minutes at 1100×g. For comparison, 1.0 mL of human buffycoat (a plasma-white blood cell mixture tinged with red blood cells) was added to 7.0 mL of neat STEEN™ and spun for 10 minutes at 1100×g in an 8.5 mL PPT tube. Physical observations included a finding of clarification after centrifugation of fluid-phase above the gel plug and migration below the plug of the cell fraction. The observed thin line at the top of the gel after separation was due to embedding to red blood cell fragments in the gel material, a phenomenon also seen when spinning whole blood into a plasma separator gel.

The experiment used a controlled buffy coat spike into STEEN solution to simulate a substantial leukocyte/RBC complement that may be present within the STEEN solution circulating through ex vivo lung perfusion (EVLP) lungs. Migration of cells through the gel separator to form a small red-tinged pellet at the bottom of the PPT below the gel separator indicates that cells suspended in STEEN™ solution pass through the gel plug of PPTs to form a pellet well-separated from the fluid phase in a manner analogous to that observed during centrifugation of human whole blood samples in a PPT. This indicates that, for samples of STEEN perfusate collecting during EVLP, the post-spin supernatant poured off a PPT will be suitable for cf-DNA analysis without problematic contamination by DNA from contaminating leukocytes.

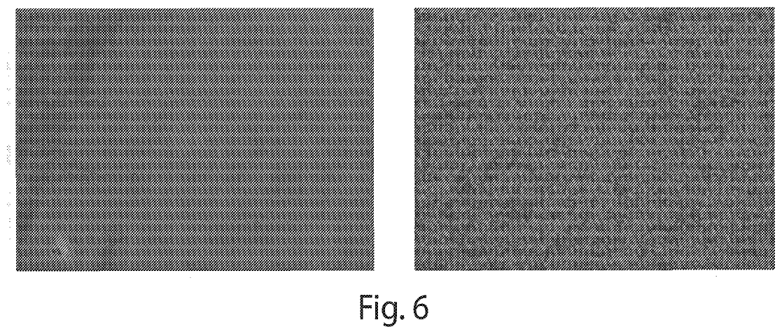

As a result, a conditional paired control step demonstrating the cellular migration of buffy coat cells spiked into purified human plasma versus STEEN solution was performed. Plasma was purified for cf-DNA extraction and analysis by centrifuging a transplant recipient's whole blood in a PPT, then pouring off and briefly recentrifuging the PPT supernatant in a conical tube at low speed to pellet any residual cellular debris. In order to evaluate the degree of clearance of leukocytes at the microscopic level from the fluid phase of intra-EVLP STEEN perfusate (as opposed to whole blood), supernatant prepared as described above was poured off into a 15 mL conical tube and subjected to a second spin at 1400×g for 10 minutes. The supernatant from the second spin was then removed from the conical tube, leaving 200 μL at the bottom of the tube to prevent disruption of the small cellular pellet. The collected supernatant was examined microscopically by hemocytometry for any remaining cellularity. The 200 μL volume at the bottom of the tube including the pellet was resuspended and also viewed microscopically through a hemocytometer. FIG. 6 shows the microscopic results.

A single low speed spin of a buffycoat-STEEN™ mixture through a gel separator PPT, followed by a clean-up low speed (1400×g for 10 minutes) spin, leaves the remaining plasma-STEEN™ fluid phase essentially completely clear of contaminating leukocytes, allowing meaningful analysis of cf-DNA in STEEN™ perfusates. The experiment demonstrates that STEEN solution and its component molecules (Dextran 40, for example) does not substantially effect the ability of the protocol for plasma purification described above to similarly remove contaminating leukocytes from STEEN-based EVLP perfusion solutes collected for cf-DNA analysis.

### Example 2—Determination of the Presence/Absence of Human DNA in Neat STEEN™

Four mL volumes of neat STEEN™ solution, a low cf-DNA positive extraction control (PEC; human plasma) and a negative extraction control (NEC; nuclease-free water) were extracted in triplicate using an automated DNA extraction workflow. Eluates from the extraction process were monitored for detection of a human reference gene by a highly sensitive PCR method validated for quantification of cf-DNA. For comparison, PCR detection assay was also applied to neat STEEN™ solution without extraction.

TABLE 1
Reference Gene Amplification

| | Evidence of Reference |
|---|---|
| | Gene Amplification |
| Sample Type | (Yes/No) |
| STEEN ™—extracted (in triplicate) | No |
| STEEN ™—not extracted (in triplicate) | No |
| NEC-nuclease-free water-1 | No |
| PEC-human plasma-1 | Yes |

No human DNA was detected in either the non-extracted STEEN™ or the extracted STEEN™. This indicates that baseline levels of human DNA in STEEN™ are at a minimum extremely low and below the level of detections for this highly sensitive assay. If STEEN™ does contain any human DNA, it is probably at a level too low to confound cf-DNA measurements.

### Example 3—Evaluation of DNA in a Neat STEEN™ Solution Using Endogenous Long and Short Fragments of DNA

Three 4 mL extractions of neat STEEN™ solution were extracted and analyzed using the short and long fragment DNA tests in triplicate, including a positive extraction control (PEC; a human plasma sample), a negative extraction control (NEC; nuclease-free water), non-extracted STEEN™ solution, and non-extracted 0.1×TE buffer.

Long and short fragment cf-DNA was detected at levels historically expected in the normal, well-characterized PEC after standard automated cf-DNA extraction. In the same run, using this more sensitive long and short fragment quantification assay, and in agreement with the results obtained in Example 2 using the reference gene qPCR assay, no DNA amplification was detected in the NEC, the non-extracted STEEN™ solution, or the extracted STEEN™ solution. Thus, the STEEN™ solution was found to not contain human DNA at a level conceivably able to confound cf-DNA evaluations.

### Example 4—Sheared Genomic DNA (gDNA) or Short Fragment gDNA Spike-In Into STEEN™ Solution Versus Human Plasma (Quantitative Detection by Reference Gene qPCR)

Genomic DNA (gDNA) was fragmented in a controlled manner by sonication and spiked into a neat STEEN™ solution (without addition of additives, such as heparin) and plasma at defined concentrations, then extracted using an automated cf-DNA extraction methodology. Resulting concentrations of cf-DNA in the extraction eluates were measured using the reference gene qPCR method in order to determine the percent recovery of spiked-in DNA, as shown in Table 2.

TABLE 2
Percent Recovery of Sheared gDNA

| | | | Sheared | Mean | |
|---|---|---|---|---|---|
| | Sheared | | gDNA | gDNA | gDNA |
| | gDNA Input | | Extraction | Extraction | Extraction |
| Sample # | (ng/mL) | Matrix | (ng/mL) | (ng/ml) | % |
| 1 | 0 | STEEN | 0.0 | 0.0 | 0% |
| 2 | 0 | STEEN | 0.0 | | |
| 3 | 0.2 | STEEN | 0.2 | 0.1 | 66% |
| 4 | 0.2 | STEEN | 0.1 | | |
| 5 | 1 | STEEN | 0.4 | 0.5 | 55% |
| 6 | 1 | STEEN | 0.7 | | |
| 7 | 2.5 | STEEN | 0.8 | 0.9 | 36% |
| 8 | 2.5 | STEEN | 1.0 | | |
| 9 | 5 | STEEN | 2.1 | 2.0 | 40% |
| 10 | 5 | STEEN | 1.9 | | |
| 11 | 5 | PLASMA | 3.0 | 3.2 | 63% |
| 12 | 5 | PLASMA | 3.3 | | |
| 13 | 15 | STEEN | 7.8 | 7.1 | 48% |
| 14 | 15 | STEEN | 6.5 | | |
| 15 | 15 | PLASMA | 8.1 | 7.8 | 52% |
| 16 | 15 | PLASMA | 7.5 | | |

Genomic DNA extraction efficiency is far from plasma or STEEN™ solution, congruent with known properties of extraction methodologies of multiple types, although the chemistry used for cf-DNA extraction was selected to be optimally efficient for efficient translation of short fragmented DNA (e.g., cf-DNA). Recognizing that normal plasma matrix has a low baseline content of cf-DNA, the data in Table 2 demonstrates that the automated cf-DNA methodology is capable of isolating total cf-DNA from human plasma.

To examine the same principles with short fragment gDNA, 25,000 copies of short DNA fragment control were spiked into samples of STEEN™ solution and human plasma containing a background of varying amounts of sheared human gDNA amounts, then subjected to an automated cf-DNA extraction protocol. The percent recovery of the short fragment in the extraction eluate was determined using the reference gene qPCR methods. The results are shown in Table 3 below.

TABLE 3
Percent Recovery of Short Fragment gDNA

| | Sheared | | Short | |
|---|---|---|---|---|
| | gDNA | | Fragment | Short |
| | Input | | Extraction | Fragment |
| Sample # | (ng/mL) | Matrix | (copies/mL) | Extraction % |
| 1 | 0 | STEEN | 14448 | 58% |
| 2 | 0 | STEEN | 9655 | 39% |
| 3 | 0.2 | STEEN | 14868 | 59% |
| 4 | 0.2 | STEEN | 14710 | 59% |
| 5 | 1 | STEEN | 14310 | 57% |
| 6 | 1 | STEEN | 11757 | 47% |
| 7 | 2.5 | STEEN | 13961 | 56% |
| 8 | 2.5 | STEEN | 15726 | 63% |
| 9 | 5 | STEEN | 11627 | 47% |
| 10 | 5 | STEEN | 12618 | 50% |
| 11 | 5 | PLASMA | 16787 | 67% |
| 12 | 5 | PLASMA | 16768 | 67% |
| 13 | 15 | STEEN | 14723 | 59% |
| 14 | 15 | STEEN | 14835 | 59% |
| 15 | 15 | PLASMA | 17131 | 69% |
| 16 | 15 | PLASMA | 14710 | 59% |
| 17 | PEC | PLASMA | 0 | 0% |
| 18 | NEC | NFW | 0 | 0% |

Recognizing that normal plasma matrix has a low baseline content of cf-DNA, the data in Table 3 demonstrate that the automated cf-DNA extraction methodology is capable of isolating short DNA fragments from STEEN™ solutions with efficiency similar to that observed when extracting from human plasma. This is beneficial for the determination of cellular apoptosis from cellular lysis during the processing of EVLP samples.

### Example 5-Evaluation of the Degree of PCR Inhibition by Heparin at Concentrations Used in the EVLP System

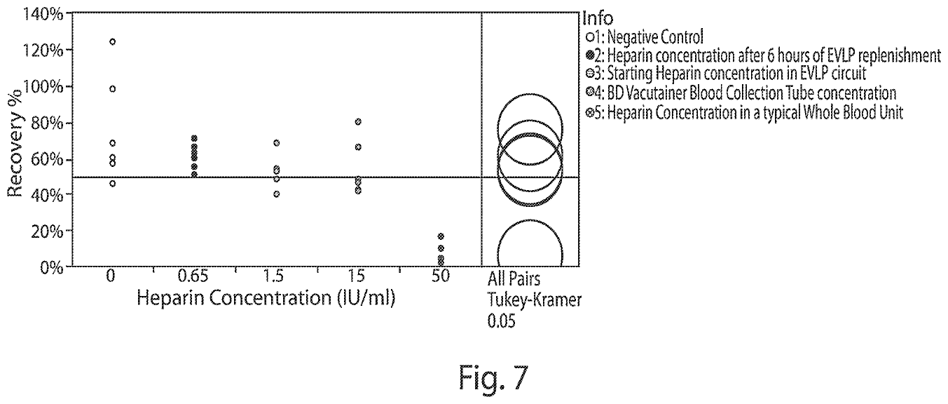

gDNA and heparin were added to STEEN™ solution at various concentrations up to 50 IU/mL. cf-DNA was extracted and quantified using an automated extraction system, and extraction efficiency was measuring using the reference gene qPCR. The results are shown in FIG. 7.

Inhibition of PCR, as measured by the quantification of a well-known reference gene is noticeable at approximately 50 IU/mL of heparin, a concentration of heparin typically used in whole blood unit donation bags. At this high concentration of heparin, recovery of the genomic reference gene drops dramatically, as shown in FIG. 7. Concentrations of heparin similar to blood collection tubes (approximately 15 IU/mL), concentrations of heparin the perfusate at the start of the EVLP circuit (approximately 1.5 IU/mL), and those after 6 hours of EVLP replenishment (approximately 0.65 IU/mL) are well below concentrations of heparin that would significantly inhibit PCR.

### Example 6—Quantification of Human DNA in Ex Vivo Lung Perfusion (EVLP) Perfusates as Measured by a Reference Gene qPCR Method

Two mL volumes from initial Tubes 1-5 were subjected to automated DNA extraction after zero (Sample Level 1), one (Sample Level 2A), or two (Sample Level 3) 1100×g×10 min spins to remove cells and debris. The extracted DNA eluates were analyzed for total DNA concentration using a reference gene qPCR method. Results are shown in Table 4.

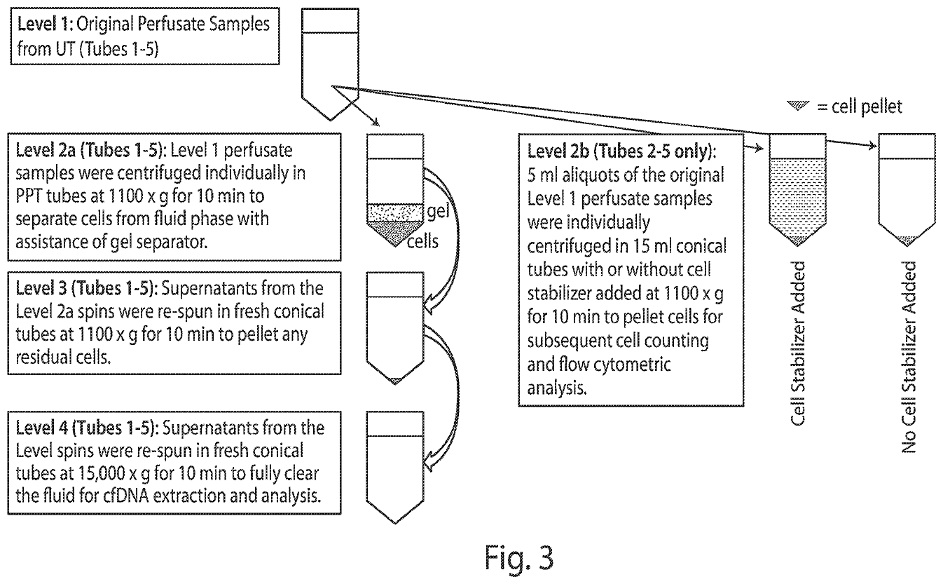

TABLE 4
Total Extracted DNA Concentration (ng/mL;
mean of duplicate determinations)

| Processing Level (Refer to | Tube | Mean Total DNA (ng/ml |
|---|---|---|
| FIG. 3) | Number | extracted) |
| 1 (original unspun tube) | Tube 1 | Undetectable |
| | Tube 2 | 909 |
| | Tube 3 | 1924 |
| | Tube 4 | 856 |
| | Tube 5 | 1094 |
| 2a (supernatant after PPT | Tube 2 | 383 |
| tube spin) | Tube 3 | 984 |
| | Tube 4 | 586 |
| | Tube 5 | 556 |
| 3 (supernatant after | Tube 2 | 462 |
| additional 1100 g spin) | Tube 3 | 1052 |
| | Tube 4 | 429 |
| | Tube 5 | 472 |

No human DNA was detected in tube 1, consistent with no exposure within the perfusion circuit to a human lung. However, relatively high concentrations of human DNA (856-1924 ng/ml) were detected in tubes 2-5 prior to centrifugation (Level 1). With the progressive removal of intact nucleated cells from the fluid phase by centrifugation (Levels 2a and 3), the concentrations of total DNA present were predictably reduced, primarily as the result of the Level 2a spin that removed most of the cells, but remained relatively high in the cell-free supernatants of Levels 2a and 3.

The concentrations of cf-DNA observed in perfusate samples 2-5 (Levels 2a and 3) are notably elevated compared to, for instance, normal circulating cf-DNA levels historically observed in plasma from normal human subjects and most patients with heart transplant rejection. However, as noted in Example 7, the samples were shipped overnight without the essential spin within 2 hours of collection to separate cells from the fluid phase, where the true cf-DNA of interest is contained. For these current EVLP samples, for which centrifugation was not possible until after the overnight shipment was received, the high “cf-DNA” levels measured could have resulted from either post-collection leukocyte lysis or from cellular lysis occurring during EVLP.

### Example 7—Cell Content of Perfusate Samples as a Function of Centrifugal Purification, Assessed by Automated Cell Count (Cell-Dyne)

Supernatant aliquots were collected from Tubes 1-5 after zero (level 1), one (level 2A), and two (level 3) 1100×g low speed spins. Cell count analyses on these aliquots were performed using a Cell-Dyne 3700 Hematology Analyzer (see Table 5).

TABLE 5
Cell count analyses (RBC/mL and WBC/mL) for Tubes 1-5

| Processing Level | Tube | | |
|---|---|---|---|
| (Refer to FIG. 3) | Number | RBC/mL | WBC/mL |
| 1 (original unspun | Tube 1 | Undetectable | Undetectable |
| tube) | Tube 2 | 20670000 | 35640 |
| | Tube 3 | 27680000 | 43790 |
| | Tube 4 | 1800000 | 16840 |
| | Tube 5 | 1700000 | 16390 |
| 2a (supernatant | Tube 2 | 100000 | 4730 |
| after PPT tube spin) | Tube 3 | 200000 | 2750 |
| | Tube 4 | Undetectable | 2640 |
| | Tube 5 | Undetectable | 3190 |
| 3 (supernatant after | Tube 2 | Undetectable | Undetectable |
| additional 1100 g | Tube 3 | 100000 | 110 |
| spin) | Tube 4 | Undetectable | Undetectable |
| | Tube 5 | Undetectable | Undetectable |

Cell count analysis shows close similarity, within the inherent variability of the Cell-Dyne method at these cell count levels, of Tubes 2 and 3, and of Tubes 4 and 5. These similarities are matched by comparisons of the appearances of the initially received tubes and the post-centrifugation tubes.

Importantly, centrifugation of the perfusate samples through levels 2a and 3 was found to be extremely effective in removing intact, countable cells. However, the slight residuum of RBCs and WBCs in one of the most cellular tubes (Tube 3) suggests a third spin may be beneficial prior to shipment for analysis.

### Example 8—Evaluation of cf-DNA Fragmentation in EVLP Perfusate Samples

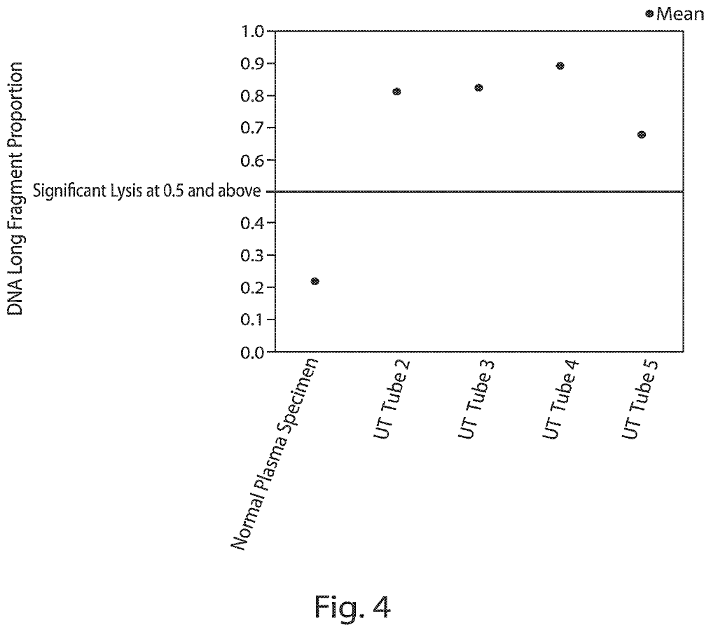

DNA was extracted from duplicate 2 mL aliquots of pristine level 4 supernatants of Tubes 2-5 and a normal plasma control specimen using automated extraction technology. The extraction eluates were analyzed using a method for differentially detecting long and short fragments of DNA as a measure of the differential contributions of cellular apoptosis (the typical mode of cellular death in vivo that produces very short DNA fragments) versus cellular lysis typically occurring during sample processing (which produces longer DNA fragments). The proportion of long fragment DNA in the eluates (mean of duplicate determinations) is shown in FIG. 4.

Extracted DNA in the level 4, acellular supernatants prepared from UT tubes 2-5 contains a high proportion of long fragment DNA that exceeds the level which was previously determined to indicate a significant degree of leukocyte lysis which would contaminate the true cf-DNA complement of the fluid phase of the specimen with lysed leukocyte DNA. This would significantly complicate interpretation of cf-DNA levels as a measure of ex vivo lung injury during perfusion, unless those leukocytes and any other intact cells are removed quickly after sample collection by 2-3 short centrifugation steps at prior to shipment for analysis.

### Example 9—Evaluation of Overall DNA Fragment Length Distribution in EVLP Perfusate Samples (Bioanalyzer Micro-Capillary-Based Electrophoresis)

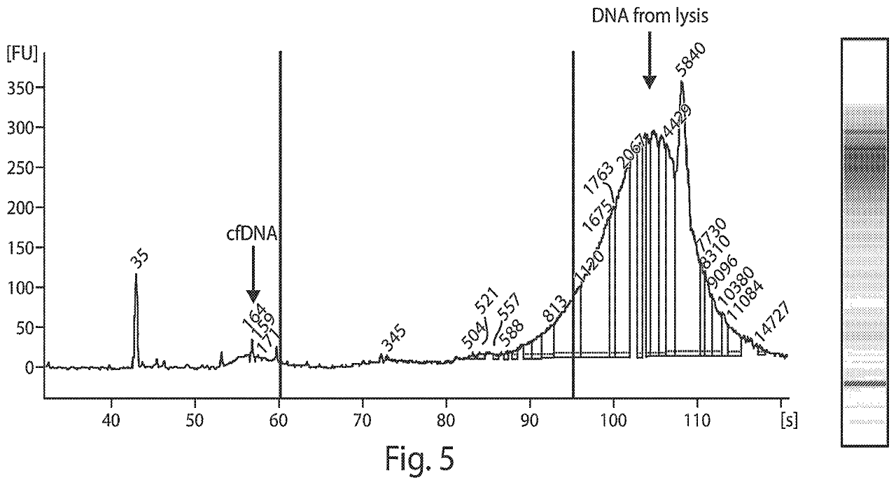

Level 4 perfusate DNA from Tubes 2-5 was extracted by an automated extraction system. 1 μl of extracted eluate was loaded onto an Agilent High Sensitivity DNA Bioanalyzer chip, and run on TAI's Bioanalyzer 2100. This micro-capillary-based electrophoretic cell allows rapid and sensitive investigation of DNA fragment length distribution. A representative tracing is shown in FIG. 5.

The representative electropherogram and gel image in FIG. 5 shows a small peak of cf-DNA around 150 bp that is consistent with apoptotic DNA release, and a much larger peak extending from 500-15,000 bp. This 500-15,000 bp peak indicates marked cell lysis in the perfusate sample and independently confirms the findings of the long fragment proportion test (see Example 3).

### Example 10—Evaluation of Recovery Efficiency of a Short DNA Fragment Control Spiked Into Pre-EVLP STEEN™

25,000 copies of a short DNA fragment control, and 15 ng/ml sheared human gDNA (sheared to an average of 150 bp in length) were spiked into an aliquot of Tube 1 (level 1) fluid, which had been determined in Examples 1 and 2 to be acellular and without detectable endogenous DNA. DNA was extracted from this spiked sample of Tube 1 using an automated DNA extraction protocol, and the percent recovery of the short fragment DNA control and sheared gDNA in the DNA extraction eluate was determined using reference gene qPCR methods. Additionally, a short vs long DNA fragment assay was used to determine the DNA long fragment proportion of the spiked sample, which was expected to be low based on the shearing protocol employed in the experimental design. Results are shown below in Tables 6-8.

TABLE 6
Percent Recovery of Short Fragment DNA Control after Extraction

| Processing Level (Refer to | Sample | Mean Short Fragment DNA |
|---|---|---|
| FIG. 3) | Name | Control Recovery % |
| 1 | Tube 1 | 47% |

TABLE 7
Percent Recovery of Spiked Sheared gDNA after Extraction

| Processing Level (Refer | Sample | Mean Sheared gDNA |
|---|---|---|
| to FIG. 3) | Name | Recovery % |
| 1 | Tube 1 | 50% |

TABLE 8
Long Fragment DNA Proportion of Spiked
Sheared gDNA after Extraction

| Processing Level | Sample | Mean Sheared gDNA Long |
|---|---|---|
| (Refer to FIG. 3) | Name | Fragment Proportion |
| 1 | Tube 1 | 0.10 |

The data presented in Tables 6-8 demonstrates that the automated cf-DNA extraction methodology utilized performs well in extracting short DNA fragments (which are typical of apoptotic cf-DNA) from pre-EVLP STEEN solution-containing standard EVLP additives. The extraction efficiency is comparable to that historically seen when extracting short fragment DNA from human plasma samples using the same method. As a general rule, short DNA fragments are not as easily extracted as long DNA fragments by any DNA extraction method, but the instant procedure has been optimized to do this well and it does so from STEEN solutions as well as from plasma.

### Example 11—Immunophenotypic Identification of Contaminating Cell Types in EVLP Perfusate Samples (Flow Cytometry)

Five ml aliquots from level 1 Tubes 2-5 were transferred per the sample processing protocol shown in FIG. 3 into 15 ml conical tubes and centrifuged for 10 minutes at 1100×g (Level 2b). Supernatants were removed and pellets were resuspended in 500 μl PBS for flow cytometric analysis. Results are shown below for each tube.

Tube 2.

| Population | #Events | % Parent | % Total |
|---|---|---|---|
| [symbol] All Events | 13,698 | #### | 100.0 |
| [symbol] WBCs | 6,402 | 46.7 | 46.7 |
| ▭ Lymphocytes | 2,099 | 32.8 | 15.3 |
| [symbol] CD4+ CD3+ | 323 | 15.4 | 2.4 |
| [symbol] CD8+ CD3+ | 369 | 17.6 | 2.7 |
| [symbol] CD56+ | 1,144 | 54.5 | 8.4 |
| [symbol] CD56+ CD3+ | 44 | 2.1 | 0.3 |
| [symbol] CD3+ | 677 | 32.3 | 4.9 |
| [symbol] CD19+ | 155 | 7.4 | 1.1 |
| [symbol] Monocytes | 172 | 2.7 | 1.3 |
| [symbol] Granulocytes | 2,288 | 35.7 | 16.7 |

Tube 3.

| Population | #Events | % Parent | % Total |
|---|---|---|---|
| [symbol] All Events | 22,560 | #### | 100.0 |
| [symbol] WBCs | 12,419 | 55.0 | 55.0 |
| ▭ Lymphocytes | 4,127 | 33.2 | 18.3 |
| [symbol] CD4+ CD3+ | 884 | 21.4 | 3.9 |
| [symbol] CD8+ CD3+ | 654 | 15.8 | 2.9 |
| [symbol] CD56+ | 1,790 | 43.4 | 7.9 |
| [symbol] CD56+ CD3+ | 65 | 1.6 | 0.3 |
| [symbol] CD3+ | 1,526 | 37.0 | 6.8 |
| [symbol] CD19+ | 591 | 14.3 | 2.6 |
| [symbol] Monocytes | 589 | 4.7 | 2.6 |
| [symbol] Granulocytes | 4,693 | 37.8 | 20.8 |

Tube 4.

| Population | #Events | % Parent | % Total |
|---|---|---|---|
| [symbol] All Events | 14,601 | #### | 100.0 |
| [symbol] WBCs | 6,928 | 47.4 | 47.4 |
| ▭ Lymphocytes | 2,323 | 33.5 | 15.9 |
| [symbol] CD4+ CD3+ | 910 | 39.2 | 6.2 |
| [symbol] CD8+ CD3+ | 434 | 18.7 | 3.0 |
| [symbol] CD56+ | 546 | 23.5 | 3.7 |
| [symbol] CD56+ CD3+ | 55 | 2.4 | 0.4 |
| [symbol] CD3+ | 1,316 | 56.7 | 9.0 |
| [symbol] CD19+ | 283 | 12.2 | 1.9 |
| [symbol] Monocytes | 112 | 1.6 | 0.8 |
| [symbol] Granulocytes | 2,547 | 36.8 | 17.4 |

Tube 5.

| Population | #Events | % Parent | % Total |
|---|---|---|---|
| [symbol] All Events | 13,806 | #### | 100.0 |
| [symbol] WBCs | 5,970 | 43.2 | 43.2 |
| ▭ Lymphocytes | 2,272 | 38.1 | 16.5 |
| [symbol] CD4+ CD3+ | 881 | 38.8 | 6.4 |
| [symbol] CD8+ CD3+ | 442 | 19.5 | 3.2 |
| [symbol] CD56+ | 564 | 24.8 | 4.1 |
| [symbol] CD56+ CD3+ | 57 | 2.5 | 0.4 |
| [symbol] CD3+ | 1,304 | 57.4 | 9.4 |
| [symbol] CD19+ | 264 | 11.6 | 1.9 |
| [symbol] Monocytes | 105 | 1.8 | 0.8 |
| [symbol] Granulocytes | 2,072 | 34.7 | 15.0 |

The tables generated by the flow cytometry analysis for Tubes 2-5 show the samples to be comparable in cellular composition as revealed by a standard human leukocyte antibody panel. This is consistent with the cellular morphology seen in matching cytospin preparations.

### Example 12—Materials and Methods (Examples 6-11)

Five de-identified, uncentrifuged samples from human ex vivo lung perfusion (EVLP) procedures were obtained (initial Tubes 1-5). The EVLP procedure used included a gradual rewarming of the lungs to normal core body temperature in conjunction with a gradual increase in vascular flow, targeting a perfusion flow of 40% donor-predicted cardiac output (CO) (Machuca et al., J Thorac Dis. 2014, 6(8):1054-1106). Protective lung ventilation and an acellular perfusate with increased colloid osmotic pressure were attained through the use of human serum albumin and Dexran 40. The methodology has been FDA-approved under a humanitarian device exemption (HDE). During EVLP, the perfusion circuit of the lung mimics in vivo conditions. The ventilated ex vivo lungs are perfused with STEEN™ solution without red blood cells. Parameters, such as gaseous exchange, pulmonary vascular resistance, compliance, and other key variables under normothermic conditions are monitored. Six hours or more of EVLP is clinically considered the standard when using an acellular STEEN perfusate. STEEN™ solution, a buffered extracellular solution includes human serum albumin for osmotic pressure and Dextran 40, a mild scavenger used to coat and protect the endothelium from excessive leukocyte interaction (Steen et al., Lancet 2001, 357:825-829; Steen et al., Ann Thorac Surg. 2003, 76:244-252; Steen et al., Ann Thorac Surg. 2007; 83:2191). The solution is designed to facilitate prolonged evaluation of lung transplantation options and to promote health of the isolated lungs ex vivo. EVLP using the STEEN™ solution thus has the potential to be able to increase the likelihood that previously rejected, but ex vivo rehabilitated lungs could be used to increase the availability of potential organs for lung transplantation.

The five samples were collected from two human lung perfusion procedures. The samples were shipped, unprocessed, overnight with cold packs. The samples were never frozen. Sample details are provided in Table 9 below.

TABLE 9
EVLP Sample Collection Details

| | Tube# | | | | |
|---|---|---|---|---|---|
| | 1 | 2 | 3 | 4 | 5 |
| Amount | 30 ml | 30 ml | 30 ml | 30 ml | 30 ml |
| Perfusate | LPD-2A | Single | Single | Single | Single |
| type | (Steen) | right | right | right | right |
| Timing | Before | After 1 | After 2 | After 1 | After 2 |
| | Lungs | hour | hour | hour | hour |
| | perfusion. | perfusion, | perfusion, | perfusion, | perfusion, |
| | LPD-2A | before | before | before | before |
| | was | exchange | exchange | exchange | exchange |
| | Primed in | | | | |
| | circuit. No | | | | |
| | contact | | | | |
| | to lungs. | | | | |
| Prime | 2000 ml | 2000 ml | 2000 ml | 2000 ml | 2000 ml |
| amount | | | | | |
| Addi- | Heparin | | 500 ml | | 500 ml |
| tional | 3000 IU, | | exchange | | exchange |
| infor- | Imipenum | | after | | after |
| mation | 500 mg, | | sampling | | sampling |
| | Solumedrol | | | | |
| | 500 mg | | | | |

The five samples were subjected to the centrifugation steps outlined in FIG. 3. The resulting supernatants and cellular pellets were used in the Experiments described below.

For the cellular analyses, as indicated in the right half of FIG. 3 (level 2B), 5 mL aliquots from each of four received tubes (Tubes 2-5 from level 1) were transferred into two individual 15 mL conical tubes spiked with either a cellular preservative or no preservative. Tubes were centrifuged at 1100×g for 10 minutes. Tubes 2 and 3 and Tubes 4 and 5 showed similar appearances. The resulting level 2b supernatants were removed, and the remaining cell pellets were resuspended in 0.5 mL phosphate buffered saline (PBS) for flow cytometry and cell counting.

For cf-DNA extraction and fragment analysis, shown as level 2a in FIG. 3, 8.5 mL aliquots from each of the received tubes (Tubes 1-5 of level 1) were transferred from those tubes into similarly labeled individual PPT tubes. The PPT tubes were then centrifuged at 1100×g for 10 minutes. Tube 1 yielded no cellular pellet. A 500 μL aliquot of each level 2a supernatant was collected for cell counting analysis (Cell Dyne) and the remaining approximately 8 mL of supernatant was transferred to a fresh 15 mL conical tube that was then subjected to a second low speed spin at 1100×g for 10 minutes (level 3 of FIG. 3). There was a lack of a visually obvious residual cellular pellet following this spin step.

Aliquots of the level 3 supernatants were collected (500 μL) for cell count analysis, flow cytometer, and initial RNAseP DNA quantification. Avoiding the last 500 μL in the tube's bottom tip, the remaining supernatants were transferred to a fresh 15 mL conical tubes and centrifuged at 15,000×g for 15 minutes. The resultant pristine level 4 supernatants (FIG. 3) were collected for cf-DNA extraction and quantification/fragment analysis.

1-35. (canceled)

36. A method of preparing a preparation of amplified nucleic acids from a sample comprising cell-free nucleic acids released from a potential graft and assessing the suitability of the potential graft for transplantation or implantation, comprising:
(a) extracting cell-free nucleic acids from the sample, wherein the sample is obtained from a perfusate in contact with the potential graft ex vivo and comprises cell-free nucleic acids released from the potential graft ex vivo, wherein the perfusate is a buffered extracellular solution comprising Dextran 40 and human serum albumin, wherein the potential graft is a lung, and wherein the sample is processed with a step that removes contaminating cells from the sample prior to extracting cell-free nucleic acids,
(b) amplifying the cell-free nucleic acids extracted in (a), and
(c) assessing the amplified nucleic acids to quantify an amount of graft-specific cell-free nucleic acids to determine the suitability of the potential graft for transplantation or implantation, and
wherein the method further comprises quantifying short nucleic acids fragments having 75-170 base pairs in length which are indicative of cell apoptosis and long nucleic acids fragments having 500-15,000 base pairs in length which are indicative of cell lysis.

37. The method of claim 36, wherein the method further comprises obtaining the potential graft from a donor.

38. The method of claim 36, wherein the method further comprises obtaining an amount of graft-specific cell-free nucleic acids at one or more additional time points.

39. The method of claim 36, wherein the method further comprises comparing the amount of graft-specific cell-free nucleic acids with a threshold value or a value determined from one or more previous time points.

40. The method of claim 36, wherein the potential graft is monitored over time.

41. The method of claim 36, wherein step (c) comprises quantifying the amount of graft-specific cell-free nucleic acids by performing real-time PCR or digital PCR.

42. The method of claim 36, wherein step (c) comprises quantifying the amount of graft-specific cell-free nucleic acids by performing next-generation sequencing.

43. The method of claim 36, wherein the amount of graft-specific cell-free nucleic acids above a threshold value or a value determined from a previous time point is indicative of decreasing suitability, or unsuitability, of the graft.

44. The method of claim 36, wherein the amount of graft-specific cell-free nucleic acids below a threshold value or a value determined from a previous time point is indicative of increasing suitability, or suitability, of the graft.

45. The method of claim 36, wherein step (b) comprises performing mismatch PCR amplification to obtain the amplified nucleic acids.

46. The method of claim 36, wherein the potential graft is obtained from a different species than human.

47. The method of claim 36, wherein the potential graft is obtained from a pig.

48. The method of claim 36, wherein the method further comprises quantifying the amount of cell lysis at one or more additional time points.

49. The method of claim 48, wherein the method further comprises comparing the amount of cell lysis with a threshold values or a value determined from one or more previous time points.

50. The method of claim 48, wherein amount of cell lysis is quantified by performing fragment analysis, electrophoresis, or quantitative amplification.
